(19) United States
(12) Patent Application Publication
(10) Pub. No.: US 2010/0158848 A1
(43) Pub. Date: Jun. 24, 2010

(54) COSMETIC COMPOSITION COMPRISING A BRANCHED SULPHONIC POLYESTER AND A SURFACTANT AND USES IN HAIR STYLING

(76) Inventors: Cécile BEBOT, Clichy (FR); Dorothée PASQUET, Bois Colombes (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/640,568

(22) Filed: Dec. 17, 2009

Related U.S. Application Data
(60) Provisional application No. 61/144,220, filed on Jan. 13, 2009.

(30) Foreign Application Priority Data
Dec. 17, 2008 (FR) 0858677

(51) Int. Cl. A61K 8/85; A61Q 5/06

(52) U.S. Cl. 424/70.9; 424/70.11

(57) ABSTRACT

The present application relates to a cosmetic composition comprising, in a cosmetically acceptable medium: at least one branched sulphonic polyester, and at least one non-ionic oxyalkylenated or glycerolated surfactant. The present application also relates to the uses of this composition, for example for the styling and the shaping of the hair.

This application claims benefit of U.S. Provisional Application No. 61/144,220, filed Jan. 13, 2009. This application also claims benefit of priority under 35 U.S.C. §119 to French Patent Application No. 0858677, filed Dec. 17, 2008.

The present disclosure relates to novel cosmetic compositions comprising at least one branched sulphonic polyester and at least one non-ionic oxyalkylenated or glycerolated surfactant, and also to the uses of these compositions, for example, in styling.

The present disclosure also relates to a method of styling keratin materials using these compositions.

The use of branched sulphonic polyesters in hair styling and fixing compositions is known and described, for example, in patent applications EP 0 966 946, WO 98/38969 and WO 99/63955.

Nevertheless, the use of branched sulphonic polyesters is not without drawbacks:
the use of these polyesters in hair lacquers containing large quantities of alcohol generally gives good styling properties but does not necessarily make it possible to obtain sufficient lacquering power;
the application of these polyesters in the form of lacquers with a high alcohol content can give the hair, after brushing, a dry feel; this undesirable phenomenon is for example noticeable for dyed hair, and
these polyesters are generally in semi-solid form and this property often makes their use difficult, for example it is often difficult to ensure a homogeneous distribution of these polyesters over all the hair to be treated.

Applicant has demonstrated that the use of at least one branched sulphonic polyester with at least one non-ionic oxyalkylenated or glycerolated surfactant makes it possible to overcome at least one, and in certain embodiments all of the aforementioned drawbacks.

One subject of the present disclosure is a cosmetic composition comprising, at least one branched sulphonic polyester and at least one non-ionic oxyalkylenated or glycerolated surfactant.

The oxyalkylenated or glycerolated surfactant may be a compound comprising at least one hydrocarbon-based chain comprising at least 6 carbon atoms and at least one group of structure:

—CH₂—(C(H)ₜ(CH₂R₁)ₙ)_{q}—CH₂p-O—

wherein n, p, and q represent, independently of one another, 0 or 1;

t represents 1 or 2; and

R₁ representing a hydrogen atom or a hydroxy radical;

the composition comprises at least one non-ionic oxyalkylenated or glycerolated surfactant ranging from 0.1 to 3% by weight, relative to the total weight of the composition.

The compositions obtained may be in the form of gels, mousses, sprays, creams, or pastes.

The compositions according to the present disclosure may be easy to prepare and to apply. They remain satisfactorily localized, without runs, at the point of application. The compositions according to the present disclosure may be applied without a reduction in viscosity over time.

Moreover, the compositions according to the present disclosure make it possible to give the hairstyle a natural and long-lasting form retention.

Another subject of the present disclosure is a method for styling keratin materials, for example human keratin materials such as the hair, which uses the compositions according to the disclosure.

Another subject of the present disclosure is the use of the compositions according to the disclosure, for example for styling and shaping of keratin materials, such as human keratin materials such ashair.

Other features, aspects, subjects and advantages of the disclosure will appear even more clearly on reading the description and examples that follow.

The term “(meth)acrylic” within the meaning of the present application is understood to mean “acrylic or methacrylic”.

The branched sulphonic polyesters used in the compositions of the present disclosure are known in the prior art. Their structure and synthesis are described in documents WO 95/18191, WO 97/08261 and WO 97/20899.

For example branched sulphonic polyesters may be obtained by the polycondensation of:

(a) at least one dicarboxylic acid that does not bear a sulphonic functional group,

(b) at least one diol or a mixture of a diol and a diamine,

(c) at least one monomer comprising two identical or different reactive functional groups chosen from hydroxyl, amino, and carboxyl groups, and that bears, in addition, at least one sulphonic functional group, and

(d) at least one monomer comprising at least three identical or different reactive functional groups chosen from hydroxyl, amino, and carboxyl groups.

The dicarboxylic acids that form the units (a) may be aliphatic dicarboxylic acids, alicyclic dicarboxylic acids, aromatic dicarboxylic acids, and mixtures of such acids.

Mention may be made, by way of example, of 1,4-cyclohexanedioic acid, succinic acid, glutaric acid, adipic acid, azelaic acid, sebacic acid, fumaric acid, maleic acid, 1,3-cyclohexanedioic acid, phthalic acid, terephthalic acid, and isophthalic acid and mixtures of such acids.

The diols that form the units (b) are chosen, for example, from alkanediols and polyalkylenediols, and mention may be made, by way of example, of ethylene glycol, propylene glycol, diethylene glycol, triethylene glycol and polypropylene glycol.

The diamines capable of forming one part of the units (b) may be chosen from alkanediamines and polyoxyalkylene diamines.

The expression “sulphonic functional group” of the units (c) encompasses both the sulphonic acid functional group (—SO₃H) and the corresponding salified functional groups obtained by neutralization of the sulphonic acid functional group with a base, for example an alkali metal hydroxide.

The sulphonic functional groups are for example in a form which is neutralized by an organic or inorganic base.

The units (c) are derived, for example, from dicarboxylic acids, dicarboxylic acid esters, glycols and hydroxy acids, all bearing at least one sulphonic group, in acid and/or neutralized form, for example in neutralized form.

The units (c) bearing at least one sulphonic functional group represent, for example, from 2 to 15 mol % of the total amount of monomers.

The units (d) derived from multifunctional monomers represent, for example from 0.1 to 40 mol % relative to the total amount of monomers.

The multifunctional monomers forming the units (d) are chosen, for example, from trimethylolethane, trimethyloipropane, glycerol, pentaerythritol, sorbitol, trimellitic anhydride, erythritol, threitol, dipentaerythritol, pyromellitic dianhydride and dimethylpropionic acid.

The branched sulphonic polyesters may comprise, in addition to the four types of units (a) to (d) described above, units (e) derived from monomers comprising two different reactive functional groups, chosen for example from hydroxy carboxylic acids, and amino carboxylic acids or mixtures thereof.

These units (e) may represent, for example, up to 40 mol % of the total amount of monomers (a), (b), (c), (d) and (e).

The branched sulphonic polymers used in the present disclosure can be obtained from a mixture of monomers in which the number of equivalents of carboxylic acid functional groups is substantially equal to the number of equivalents of hydroxyl functional groups and of amino functional groups which may be present.

The branched sulphonic polymers used in the styling compositions of the present disclosure are known and sold, for example, by Eastman. Mention may be made, as a possible commercial product, of the product sold under the name AQ 1350® by Eastman.

The composition according to the present disclosure can comprise the at least one branched sulphonic polyester in an amount ranging from 0.2 to 15% by weight, relative to the total weight of the composition, such as an amount ranging from 0.5 to 10% by weight.

The composition according to the present disclosure also comprises at least one non-ionic oxyalkylenated or glycerolated surfactant.

The expression “oxyalkylenated or glycerolated surfactant” is understood to mean, within the meaning of the present disclosure, a compound comprising at least one hydrocarbon-based chain comprising at least 6 carbon atoms and at least one group of structure:

—CH₂—(C(H)ₜ(CH₂R₁)n)_{q}—CH₂p-O—

wherein n, p, and q represent, independently of one another, 0 or 1

t represents 1 or 2

and R₁ represents a hydrogen atom or a hydroxy radical.

These groups may be referred to as oxyethylenated (q=0, p=1), oxypropylenated (q=1, n=0 t=2 p=1 or q=1 t=1 n=1 R₁═H) or glycerolated (q=1, n=0 t=2 p=1 or q=1 t=1 n=1 R₁═OH).

For example, the non-ionic oxyalkylenated or glycerolated surfactant may be chosen from:
oxyalkylenated or glycerolated fatty alcohols;
oxyalkylenated alkylphenols, the alkyl chain of which is a C₈-C₁₈ alkyl chain;
oxyalkylenated or glycerolated fatty amides;
oxyalkylenated vegetable oils;
oxyalkylenated sorbitan esters of C₆-C₃₀ acids;
oxyalkylenated sucrose esters of fatty acids;
polyethylene glycol esters of fatty acids;
copolymers of ethylene oxide and of propylene oxide; and
mixtures thereof.

For example, the mean number of the at least one oxyalkylene unit may range from 2 to 150 units. The at least one oxylakylene unit may be oxyethylene or oxypropylene units or mixtures thereof.

As regards the glycerolated surfactants, they may comprise, on average, from 1 to 20 glycerol groups, such as from 1.5 to 5.

In accordance with one embodiment of the disclosure, the composition comprises at least one non-ionic surfactant chosen from oxyalkylenated or glycerolated C6-C30 alcohols.

The cosmetically acceptable medium may be aqueous.

The cosmetic composition according to the disclosure may also comprise at least one organic solvent in an amount ranging from 0.05 to 40% by weight relative to the total weight of the composition, such as an amount ranging from 1 to 20% by weight.

This organic solvent may be a C2 to C4 lower alcohol, such as ethanol, polyols, and polyol ethers such as propylene glycol, polyethylene glycol or glycerol.

The compositions according to the disclosure may also comprise at least one other cosmetically acceptable adjuvants, such as for example ionic or non-ionic surfactants other than the surfactants of the disclosure, additional thickening agents, ethoxylated or non-ethoxylated fatty alcohols, co-thickeners, penetrants, fragrances, dyes, plasticizers, buffers, and various customary adjuvants such as waxes, volatile or non-volatile silicones that are cyclic or linear or branched, and are organomodified, for example, alkoxylated or modified by amine groups or are unmodified, for example silicone gums, ceramides, pseudoceramides, plant, mineral or synthetic oils, vitamins or provitamins such as panthenol, opacifiers, reducing agents, emulsifiers, preservatives, mineral fillers, pearlescent agents, flakes, sunscreens, proteins, anionic, non-ionic, cationic or amphoteric fixing polymers, moisturisers, emollients, demulcents, anti-foaming agents, antiperspirants, free-radical scavengers, bactericides, sequestrants, anti-dandruff agents, antioxidants, basifying agents, acidifying agents, and any other additive conventionally used in cosmetic compositions intended to be applied to the hair.

The additional gelling agents and/or thickeners suitable for the compositions of the disclosure are well known in the art and may be chosen from poly(oxyalkylene) glycols, poly(oxyalkylene) glycol esters, alginates, biosaccharides, starch derivatives, natural gums such as xanthan gun, guar gum, carob bean gum, scleroglucans, derivatives of chitin and of chitosan, carrageenans, clays, and mixtures thereof.

By way of example of gelling agents, such as those that are in the aqueous phase, mention may be made of SEPIGEL® 305 sold by the company SEPPIC, FUCOGEL® 1000 PP sold by the company Solabia, SYNTHALEN® K sold by the company 3VSA, LUVISKOL® VA 64 P sold by BASF, HOSTACERIN® AMPS sold by Clariant, LUBRAGEL® MS sold by Guardian, SATIAGEL® KSO sold by Degussa and KELTROL® sold by the company Kelco.

The additional gelling agents can be represent in an amount ranging from 0.05 to 15% by weight relative to the total weight of the composition, such as an amount ranging from 0.5 to 10% by weight.

The silicones that may be used as additives in the cosmetic compositions of the present disclosure are volatile or non-volatile, cyclic, linear or branched silicones, optionally modified with organic groups, having a viscosity from 5×10−6 to 2.5 m2/s at 25° C. such as 1×10−5 to 1 m2/s.

The silicones that can be used in accordance with the disclosure may be soluble or insoluble in the composition and for example may be polyorganosiloxanes that are insoluble in the composition of the disclosure. They may be in the form of oils, waxes, resins, or gums.

The organopolysiloxanes are defined in greater detail in Walter Noll's “Chemistry and Technology of Silicones” (1968), Academic Press. They can be volatile or non-volatile.

When they are volatile, the silicones may be chosen from those having a boiling point ranging from 60° C. to 260° C., such as:

(i) cyclic silicones comprising from 3 to 7 and for example 4 to 5 silicon atoms. These are, for example, octamethylcyclotetrasiloxane such as those sold under the name VOLATILE SILICONE® 7207 by Union Carbide or SILIBIONE® 70045 V2 by Rhodia, decamethylcyclopentasiloxane sold under the name VOLATILE SILICONE® 7158 by Union Carbide, and SILIBIONE® 70045 V5 by Rhodia, and mixtures thereof.

Mention may also be made of cyclocopolymers of the dimethylsiloxane/methylalkylsiloxane type, such as SILICONE VOLATILE® FZ 3109 sold by the company Union Carbide, of formula:

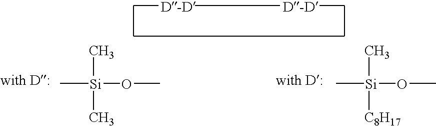

Mention may also be made of mixtures of cyclic silicones with organic compounds derived from silicon, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane.

(ii) linear volatile silicones comprising 2 to 9 silicon atoms and having a viscosity of less than or equal to 5×10⁻⁶ m²/s at 25° C. An example is decamethyltetrasiloxane such as the decamethyltetrasiloxane sold under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Non-volatile silicones, such as polyalkylsiloxanes, polyarylsiloxanes, polyalkylarylsiloxanes, silicone gums and resins, polyorganosiloxanes modified with organofunctional groups, and mixtures thereof, may be used.

These silicones may be chosen from polyalkylsiloxanes, among which mention may be made of polydimethylsiloxanes comprising trimethylsilyl end groups. The viscosity of the silicones is measured at 25° C. according to ASTM 445 standard Appendix C.

Among these polyalkylsiloxanes, mention may be made, in a non-limiting manner, of the following commercial products:
the SILIBIONE® oils of the 47 and 70 047 series or the MIRASIL® oils sold by Rhodia, such as, for example, the oil 70 047 V 500 000;
the oils of the MIRASIL® series sold by the company Rhodia;
the oils of the 200 series from the company Dow Corning, such as, DC200 with a viscosity of 60 000 mm²/s; and
the VISCASIL® oils from General Electric and certain oils of the SF series (SF 96, SF 18) from General Electric.

Mention may also be made of polymethylsiloxanes comprising dimethylsilanol end groups, known by the name dimethiconol (CTFA), such as the oils of the 48 series from the company Rhodia.

In this category of polyalkylsiloxanes, mention may also be made of the products sold under the names ABIL WAX® 9800 and 9801 by the company Goldschmidt, which are poly(C1-C20)alkylsiloxanes.

The polyalkylarylsiloxanes may be chosen from linear and/or branched polydimethyl/methylphenylsiloxanes and polydimethyl/diphenylsiloxanes with a viscosity of from 1×10−5 to 5×10−2 m2/s at 25° C.

Among these polyalkylarylsiloxanes, mention may be made, by way of example, of the products sold under the following names:

the SILIBIONE® oils of the 70 641 series from Rhodia;

the oils of the RHODORSIL® 70 633 and 763 series from Rhodia;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000; and

certain oils of the SF series from General Electric, such as SF 1023, SF 1154, SF 1250 and SF 1265.

The silicone gums that can be used in accordance with the disclosure are, for example, polyorganosiloxanes having high number-average molecular weights ranging from 200,000 to 1,000,000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Mention may be made as an example of the following products:

polydimethylsiloxane gums,

polydimethylsiloxane/methylvinylsiloxane gums,

polydimethylsiloxane/diphenylsiloxane gums,

polydimethylsiloxane/phenylmethylsiloxane gums, and

polydimethylsiloxane/diphenylsiloxane/methylvinylsiloxane gums.

Products that can be used in accordance with the disclosure may be mixtures such as:

mixtures formed from a polydimethylsiloxane hydroxylated at the end of the chain, or dimethiconol (CTFA) and from a cyclic polydimethylsiloxane also called cyclomethicone (CTFA), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSs of different viscosities, such as a PDMS gum and a PDMS oil, such as the product SF 1236 from the company General Electric. The product SF 1236 is the mixture of an SE 30 gum defined above, having a viscosity of 20 m²/s, and an SF 96 oil, with a viscosity of 5×10⁻⁶ m²/s. This product may comprise 15% SE 30 gum and 85% SF 96 oil.

The organopolysiloxane resins that can be used in accordance with the disclosure are crosslinked siloxane systems comprising the following units:

R₂SiO_{2/2}, R₃SiO_{1/2}, RSiO_{3/2} and SiO_{4/2} in which R represents a hydrocarbon-based group comprising 1 to 16 carbon atoms or a phenyl group. Among these products, R may denote a C₁-C₄ lower alkyl group, such as methyl, or a phenyl group.

Among these resins, mention may be made of the product sold under the name DOW CORNING 593 or those sold under the names SILICONE FLUID SS 4230 and SS 4267 by the company General Electric, which are silicones of dimethyl/trimethylsiloxane structure.

Mention may also be made of the trimethylsiloxysilicate type resins sold for example under the names X22-4914, X21-5034, and X21-5037 by the company Shin-Etsu.

The organomodified silicones that can be used in accordance with the disclosure are silicones as defined above and comprising in their structure at least one organofunctional group attached via a hydrocarbon-based group.

Among the organomodified silicones, mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils SILWET® L 722, L 7500, L 77, L 711 from the company Union Carbide and the (C₁₂)alkylmethicone copolyol sold by the company Dow Corning under the name Q2 5200;

substituted or unsubstituted amine groups, such as the products sold under the name GP 4 SILICONE FLUID and GP 7100 by the company Genesee, or the products sold under the names Q2 8220 and DOW CORNING 929 or 939 or DOW CORNING 2-8299 by the company Dow Corning or the product sold under the name BELSIL ADM LOG 1 by the company Wacker. The substituted amine groups may be, for example, C₁-C₄ aminoalkyl groups;

thiol groups such as the products sold under the names GP 72A and GP 71 from Genesee;

alkoxylated groups such as the product sold under the name SILICONE COPOLYMER F-755 by SWS Silicones and ABIL WAX® 2428, 2434 and 2440 by the company Goldschmidt;

hydroxylated groups such as the polyorganosiloxanes comprising a hydroxyalkyl functional group, described in French patent application FR-A-8 516 334;

alkoxyalkyl groups such as, for example, the polyorganosiloxanes described in U.S. Pat. No. 4,957,732;

anionic groups of carboxylic type, such as, for example, in the products described in patent EP 186 507 from the company Chisso Corporation, or of alkylcarboxylic type, such as those present in the product X-22-3701E from the company Shin-Etsu; 2-hydroxyalkyl sulphonate; 2-hydroxyalkyl thiosulphate such as the products sold by the company Goldschmidt under the names ABIL® S201 and ABIL® S255; and

hydroxyacrylamino groups, such as the polyorganosiloxanes described in patent application EP 342 834. Mention may be made, for example, of the product Q2-8413 from the company Dow Corning.

The silicones as described above may be used, alone or as a mixture, in an amount ranging from 0.01% to 20% by weight such as ranging from 0.1% to 5% by weight.

The compositions of the disclosure may also comprise non-silicone fatty substances such as mineral, plant, animal and synthetic oils, waxes, fatty esters, ethoxylated or non-ethoxylated fatty alcohols, and fatty acids.

As oils that can be used in the composition of the disclosure, examples that may be mentioned include:

hydrocarbon-based oils of animal origin, such as perhydrosqualene;
hydrocarbon-based oils of plant origin, such as liquid fatty acid triglycerides comprising from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names MIGLYOL® 810, 812 and 818 by the company Dynamit Nobel, jojoba oil and shea butter oil;
linear or branched hydrocarbons of mineral or synthetic origin, such as volatile or non-volatile liquid paraffins, and derivatives thereof, petroleum jelly, polydecenes, hydrogenated polyisobutene such as PARLEAM®; isoparaffins, for instance isohexadecane and isodecane; and
partially hydrocarbon-based and/or silicone-based fluoro oils, for instance those described in document JP-A-2-295 912; fluoro oils that may also be mentioned include perfluoromethylcyclopentane and perfluoro-1,3-dimethylcyclohexane, sold under the names FLUTEC® PC1 and FLUTEC® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name FORALKYL® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The wax(es) may be chosen, for example, from carnauba wax, candelilla wax, esparto grass wax, paraffin wax, ozokerite, plant waxes such as olive wax, rice wax, hydrogenated jojoba wax or the absolute waxes of flowers such as the essential wax of blackcurrant blossom sold by the company Bertin (France), animal waxes, for instance beeswaxes or modified beeswaxes (cerabellina); other waxes or waxy starting materials that can be used according to the disclosure may be marine waxes such as the product sold by the company Sophim under the reference M82, and polyethylene waxes or polyolefin waxes in general.

The saturated or unsaturated fatty acids may be chosen from myristic acid, palmitic acid, stearic acid, behenic acid, oleic acid, linoleic acid, linolenic acid, and isostearic acid.

The fatty esters may be carboxylic acid esters, such as mono-, di-, tri-, or tetracarboxylic esters.

The carboxylic acid esters may be esters of saturated or unsaturated, linear or branched C₁-C₂₆ aliphatic acids and of saturated or unsaturated, linear or branched C₁-C₂₆ aliphatic alcohols, the total carbon number of the esters being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, and 2-hexyldecyl laurate.

Esters of C₄-C₂₂ dicarboxylic or tricarboxylic acids and of C₁-C₂₂ alcohols and esters of mono-, di- or tricarboxylic acids and of C₂-C₂₆ di-, tri-, tetra-, or pentahydroxy alcohols may also be used.

The following may be mentioned: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate and neopentyl glycol diheptanoate. The esters mentioned above being different from the esters of formula (I).

Among the esters mentioned above, it is possible to use ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate, or cetyl octanoate.

As fatty alcohols, mention may be made of linear or branched, saturated or unsaturated fatty alcohols comprising from 8 to 26 carbon atoms, for instance cetyl alcohol, stearyl alcohol and the mixture thereof (cetylstearyl alcohol), octyldodecanol, 2-butyloctanol, 2-hexyldecanol, 2-undecylpentadecanol, oleyl alcohol, or linoleyl alcohol.

The fatty substances in general represent from 0.1 to 50%; such as from 1 to 30%, and such as from 2 to 20% by weight of the total composition.

As indicated previously, the compositions may comprise at least one additional fixing polymer other than the polymers of the disclosure. The expression “fixing polymer” is understood within the meaning of the present disclosure to mean any polymer that makes it possible to give a shape to the hair or to hold the hair in a given shape.

All the anionic, cationic, amphoteric and non-ionic fixing polymers and mixtures thereof used in the art may be used in the compositions according to the present application.

The fixing polymers may be soluble in the cosmetically acceptable medium or insoluble in this same medium and used in this case in the form of dispersions of solid or liquid particles of polymer (latex or pseudolatex).

The anionic fixing polymers generally used are polymers comprising groups derived from carboxylic, sulphonic or phosphoric acid and have a number-average molecular weight ranging from 500 to 5,000,000.

The carboxylic groups may be provided by unsaturated carboxylic monoacid or diacid monomers such as those corresponding to the formula:

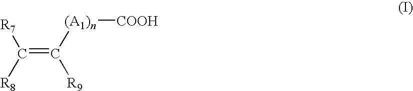

in which n is an integer from 0 to 10, A₁ represents a methylene group, optionally connected to the carbon atom of the unsaturated group or to the neighboring methylene group when n is greater than 1, via a heteroatom such as oxygen or sulphur, R₇ represents a hydrogen atom, or a phenyl or benzyl group, R₈ represents a hydrogen atom or a lower alkyl or carboxyl group, R₉ represents a hydrogen atom, a lower alkyl group or a —CH₂—COOH, phenyl or benzyl group.

In the aforementioned formula, a lower alkyl group may represent a group having 1 to 4 carbon atoms, such as methyl and ethyl groups.

The anionic fixing polymers comprising carboxylic groups that may be used according to the disclosure are:

A) The homopolymers or copolymers of acrylic or methacrylic acid or salts thereof such as the products sold under the names VERSICOL® E or K by the company Allied Colloid and ULTRAHOLD® by BASF, copolymers of acrylic acid and of acrylamide sold in the form of their sodium salts under the names RETEN 421, 423 or 425 by the company Hercules, the sodium salts of polyhydroxycarboxylic acids.

B) Copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described for example in French patent 1 222 944 and German patent application 2 330 956, the copolymers of this type comprising an optionally N-alkylated and/or hydroxyalkylated acrylamide unit in their chain as described for example in Luxembourg patent applications 75370 and 75371 or sold under the name QUADRAMER by the company American Cyanamid. Mention may also be made of the acrylic acid/ethyl acrylate/N-tert-butylacrylamide terpolymers such as ULTRAHOLD STRONG sold by the company BASF. Mention may also be made of copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by the company ISP under the name ACRYLIDONE® LM and methacrylic acid/ethyl acrylate/tert-butyl acrylate terpolymers such as the product sold under the name LUVIMER® 100 P by the company BASF.

Mention may also be made of methacrylic acid/acrylic acid/ethyl acrylate/methyl methacrylate copolymers as an aqueous dispersion, sold under the name AMERHOLD® DR 25 by the company Amerchol.

C) Crotonic acid copolymers, such as those comprising vinyl acetate or propionate units in their chain and optionally other monomers such as allyl esters or methallyl esters, vinyl ether or vinyl ester of a linear or branched saturated carboxylic acid with a long hydrocarbon chain such as those comprising at least 5 carbon atoms, it being possible for these polymers optionally to be grafted or crosslinked, or alternatively another vinyl, allyl or methallyl ester monomer of an α- or β-cyclic carboxylic acid. For example, such polymers are described, inter alia, in French Patents Nos. 1 222 944, 1 580 545, 2 265 782, 2 265 781, 1 564 110 and 2 439 798. Commercial products falling into this class may be the resins 28-29-30, 26-13-14 and 28-13-10 sold by the company National Starch.

D) Copolymers of C₄-C₈ monounsaturated carboxylic acids or anhydrides may be chosen from:
copolymers comprising (i) at least one maleic, fumaric or itaconic acids or anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described for example in U.S. Pat. Nos. 2,047,398, 2,723,248 and 2,102,113 and GB patent 839 805. Commercial products that may be used may include those sold under the names GANTREZ® AN or ES by the company ISP,
copolymers comprising (i) at least one maleic, citraconic or itaconic anhydride units and (ii) at least one monomers chosen from allyl or methallyl esters optionally comprising at least one acrylamide, methacrylamide, α-olefin, acrylic or methacrylic ester, acrylic or methacrylic acid or vinylpyrrolidone groups in their chain, the anhydride functions of these copolymers optionally being monoesterified or monoamidated.

These polymers are described, for example, in French patents 2 350 384 and 2 357 241 by the Applicant.

E) Polyacrylamides may comprise carboxylate groups.

F) Homopolymers and copolymers may comprise sulphonic groups such as polymers comprising vinylsulphonic, styrenesulphonic, naphthalenesulphonic or acrylamidoalkylsulphonic units, different from the branched sulphonic polyesters of the disclosure.

These polymers can be chosen for example from:
polyvinylsulphonic acid salts having a molecular weight of approximately between 1000 and 100,000, and also the copolymers with an unsaturated comonomer such as acrylic or methacrylic acids and their esters, and also acrylamide or its derivatives, vinyl ethers and vinylpyrrolidone;
polystyrenesulphonic acid salts such as the sodium salts that are sold for example under the names FLEXAN® 500 and FLEXAN® 130 by National Starch. These compounds are described for example in patent FR 2 198 719;
polyacrylamidesulphonic acid salts, such as those mentioned in U.S. Pat. No. 4,128,631 and for example polyacrylamidoethylpropanesulphonic acid sold under the name COSMEDIA POLYMER HSP 1180 by Henkel.

As another anionic fixing polymer that can be used according to the disclosure, mention may be made of the branched block anionic polymer sold under the name FIXATE G-100 by the company Noveon.

According to the disclosure, the anionic fixing polymers are may be chosen from copolymers of acrylic acid or of acrylic esters, such as the acrylic acid/ethyl acrylate/N-tert-butylacrylamide terpolymers sold for example under the name ULTRAHOLD® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold for example under the name RESIN 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the name GANTREZ® by the company ISP, the copolymers of methacrylic acid and of methyl methacrylate sold under the name EUDRAGIT® L by the company Rohm Pharma, the copolymers of methacrylic acid and of ethyl acrylate sold under the name LUVIMER® MAEX or MAE by the company BASF, the vinyl acetate/crotonic acid copolymers sold under the name LUVISET CA 66 by the company BASF, the vinyl acetate/crotonic acid copolymers grafted with polyethylene glycol sold under the name ARISTOFLEX® A by the company BASF, and the polymer sold under the name FIXATE G-100 by the company Noveon.

Among the anionic fixing polymers mentioned above which may be used, the context of the present disclosure, the monoesterified methyl vinyl ether/maleic anhydride copolymers sold under the name GANTREZ® ES 425 by the company ISP, the acrylic acid/ethyl acrylate/N-tert-butylacrylamide terpolymers sold under the name ULTRAHOLD® Strong by the company BASF, the copolymers of methacrylic acid and of methylmethacrylate sold under the name EUDRAGIT® L by the company Rohm Pharma, the vinylacetate/vinyl tert-butylbenzoate/crotonic acid terpolymers and the crotonic acid/vinylacetate/vinyl neododecanoate terpolymers sold under the name RESIN 28-29-30 by the company National Starch, the copolymers of methacrylic acid and of ethylacrylate sold under the name LUVIMER® MAEX or MAE by BASF, the vinylpyrrolidone/acrylic acid/laurylmethacrylate terpolymers sold under the name ACRYLIDONE® LM by the company ISP, and the polymer sold under the name FIXATE G-100 by the company Noveon.

The cationic fixing film-forming polymers that can be used according to the present disclosure may be chosen from polymers comprising primary, secondary, tertiary, and/or quaternary amine groups forming part of the polymer chain or directly attached thereto, and having a molecular weight ranging from 500 to about 5,000,000 such as ranging from 1,000 to 3,000,000.

Among these polymers, mention may be made for example of the following cationic polymers:

(1) homopolymers or copolymers derived from acrylic or methacrylic esters or amides and comprising at least one of the units of the following formulae:

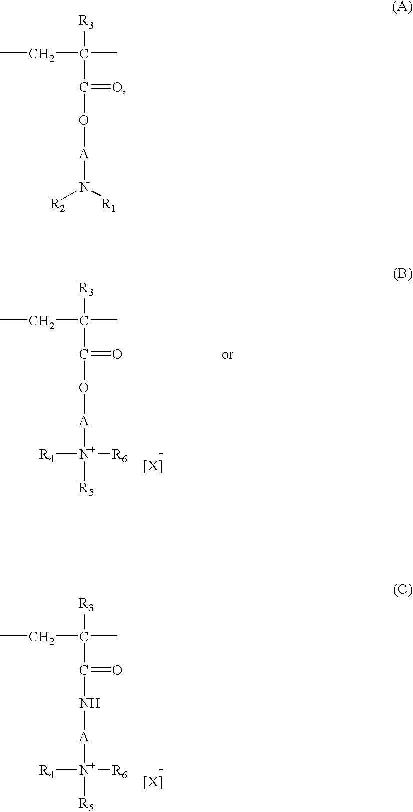

in which:

R₃ represents a hydrogen atom or a CH₃ radical;

A represents a linear or branched alkyl group comprising 1 to 6 carbon atoms or a hydroxyalkyl group comprising 1 to 4 carbon atoms;

R₄, R₅ and R₆, which may be identical or different, represent an alkyl group having from 1 to 18 carbon atoms or a benzyl radical;

R₁ and R₂, which may be identical or different, each represent a hydrogen atom or an alkyl group having from 1 to 6 carbon atoms; and

X represents a methosulphate anion or a halide such as chloride or bromide.

The copolymers of the family (1) also comprising at least one unit derived from comonomers that may be chosen from the family of acrylamides, methacrylamides, diacetone acrylamides, acrylamides and methacrylamides substituted on the nitrogen with lower (C1-4) alkyl groups, groups derived from acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulphate or with a dimethyl halide, such as the product sold under the name HEROFLOC® by the company Hercules,
copolymers of acrylamide and of methacryloyloxyethyltrimethylammonium chloride, described, for example, in patent application EP-A-080 976 and sold under the name BINA QUAT P 100 by the company Ciba Geigy,
copolymers of acrylamide and of methacryloyloxyethyltrimethylammonium methosulphate, such as the product sold under the name RETEN by the company Hercules,
quaternized or non-quaternized vinylpyrrolidone/dialkylaminoalkyl acrylate or methacrylate copolymers, such as the products sold under the name “GAFQUAT®” by the company ISP, such as, for example, “GAFQUAT® 734” or “GAFQUAT® 755”, or alternatively the products known as “COPOLYMER® 845, 958 and 937”. These polymers are described in detail in French patents 2 077 143 and 2 393 573,
fatty-chain polymers comprising a vinylpyrrolidone unit, such as the products sold under the name STYLEZE W20 and STYLEZE W10 by the company ISP,
dimethylaminoethyl methacrylate/vinylcaprolactam/vinylpyrrolidone terpolymers, such as the product sold under the name GAFFIX VC 713 by the company ISP,
quaternized vinylpyrrolidone/dimethylaminopropylmethacrylamide copolymers, such as the products sold under the name “GAFQUAT® HS 100” by the company ISP, and
quaternized homopolymers of dialkylaminoalkyl methacrylate sold under the names SALCARE SC95 or SC96 by Allied Colloids.

(2) non-cellulosic cationic polysaccharides, for example comprising quaternary ammonium, such as those described in U.S. Pat. Nos. 3,589,578 and 4,031,307, such as guar gums comprising trialkylammonium cationic groups. Such products are sold for example under the trade names JAGUAR C13S, JAGUAR C 15, and JAGUAR C 17 by the company Meyhall;

(3) quaternary copolymers of vinylpyrrolidone and of vinylimidazole;

(4) chitosans or salts thereof; the salts that can be used are, for example, chitosan acetate, lactate, glutamate, gluconate, or pyrrolidonecarboxylate.

Among these compounds, mention may be made of chitosan having a degree of deacetylation of 90.5% by weight, sold under the name KYTAN BRUT STANDARD by the company Aber Technologies, and chitosan pyrrolidonecarboxylate sold under the name KYTAMER® PC by the company Amerchol.

(5) cationic cellulose derivatives such as copolymers of cellulose or of cellulose derivatives grafted with a water-soluble monomer comprising a quaternary ammonium, and disclosed for example in U.S. Pat. No. 4,131,576, such as hydroxyalkylcelluloses, for instance hydroxymethyl-, hydroxyethyl- or hydroxypropylcelluloses optionally grafted with a methacryloyloxyethyltrimethylammonium, methacrylamidopropyltrimethylammonium or dimethyldiallylammonium salt.

The products sold corresponding to this definition are, for example, the products sold under the name “CELQUAT L 200” and “CELQUAT H 100” by the company National Starch.

The amphoteric fixing polymers that can be used in accordance with the disclosure can be chosen from polymers comprising units B and C distributed randomly in the polymer chain, in which B represents a unit derived from a monomer comprising at least one basic nitrogen atom and C represents a unit derived from an acid monomer comprising at least one carboxylic or sulphonic group, or alternatively B and C can represent groups derived from carboxybetaine or sulphobetaine zwitterionic monomers.

B and C can also represent a cationic polymer chain comprising primary, secondary, tertiary, or quaternary amine groups, in which at least one of the amine groups bears a carboxylic or sulphonic group connected via a hydrocarbon group or alternatively B and C form part of a chain of a polymer comprising an α,β-dicarboxylic ethylene unit in which one of the carboxylic groups has been made to react with a polyamine comprising at least one primary or secondary amine groups.

The amphoteric fixing polymers corresponding to the definition given above that for example may be chosen from the following polymers:

(1) copolymers having acidic vinyl units and basic vinyl units, such as those resulting from the copolymerization of a monomer derived from a vinyl compound bearing a carboxylic group such as, acrylic acid, methacrylic acid, maleic acid, α-chloroacrylic acid, and a basic monomer derived from a substituted vinyl compound comprising at least one basic atom, such as, dialkylaminoalkyl methacrylates and acrylates, dialkylaminoalkylmethacrylamides and -acrylamides. Such compounds for example described in U.S. Pat. No. 3,836,537.

(2) polymers comprising units derived from:
a) at least one monomer chosen from acrylamides and methacrylamides substituted on the nitrogen atom with an alkyl group,
b) at least one acidic comonomer comprising at least one reactive carboxylic groups, and
c) at least one basic comonomer such as esters comprising primary, secondary, tertiary, and quaternary amine substituents of acrylic and methacrylic acids and the product of quaternization of dimethylaminoethyl methacrylate with dimethyl or diethyl sulphate.

The N-substituted acrylamides or methacrylamides that may be used according to the disclosure are compounds in which the alkyl groups comprise from 2 to 12 carbon atoms such as N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers may be chosen from acrylic acid, methacrylic acid, crotonic acid, itaconic acid, maleic acid and fumaric acid and alkyl monoesters, having 1 to 4 carbon atoms, of maleic or fumaric acids or anhydrides.

The basic comonomers may be aminoethyl, butylaminoethyl, N,N′-dimethylaminoethyl, and N-tert-butylaminoethyl methacrylates.

The copolymers whose CTFA (4th edition, 1991) name is octylacrylamide/acrylates/butylaminoethyl methacrylate copolymer, such as the products sold under the name AMPHOMER® or LOVOCRYL® 47 by the company National Starch, may be used.

(3) crosslinked and acylated polyamino amides partially or totally derived from polyamino amides of general formula:

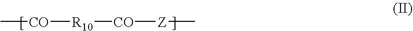

in which R₁₀ represents a divalent group derived from a saturated dicarboxylic acid, a mono- or dicarboxylic aliphatic acid comprising an ethylenic double bond, an ester of a lower alkanol, having 1 to 6 carbon atoms, of these acids, or a group derived from the addition of any one of said acids to a bis(primary) or bis(secondary) amine, and Z represents a group derived from a bis(primary), mono- or bis(secondary) polyalkylene-polyamine and for example represents:
a) in proportions ranging from 60 to 100 mol %, the group:

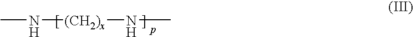

where x=2 and p=2 or 3, or alternatively x=3 and p=2
this group being derived from diethylenetriamine, from triethylenetetraamine or from dipropylenetriamine;
b) in proportions ranging from 0 to 40 mol %, the group (X) above in which x=2 and p=1 and which is derived from ethylenediamine, or the group derived from piperazine:

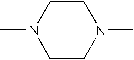

c) in proportions ranging from 0 to 20 mol %, the NH—(CH₂)₆—NH— group being derived from hexamethylenediamine,
   these polyamino amides being crosslinked by addition reaction of a difunctional crosslinking agent chosen from epihalohydrins, diepoxides, dianhydrides and bis-unsaturated derivatives, using in an amount ranging from 0.025 to 0.35 mol of crosslinking agent per amine group of the polyamino amide and acylated by the action of acrylic acid, chloroacetic acid or an alkane sultone, or salts thereof.

The saturated carboxylic acids may be chosen from acids having 6 to 10 carbon atoms, such as adipic acid, 2,2,4-trimethyladipic acid and 2,4,4-trimethyladipic acid, terephthalic acid, acids comprising an ethylenic double bond such as, for example, acrylic acid, methacrylic acid and itaconic acid.

The alkane sultones used in the acylation may be propane sultone or butane sultone; the salts of the acylating agents may be the sodium or potassium salts.

(4) polymers comprising zwitterionic units of formula:

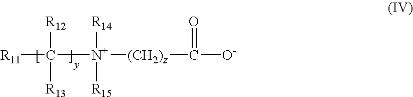

in which R₁₁ represents a polymerizable unsaturated group such as an acrylate, methacrylate, acrylamide or methacrylamide group, y and z represent an integer from 1 to 3, R₁₂ and R₁₃ represent a hydrogen atom, a methyl, ethyl, or propyl group, R₁₄ and R₁₅ represent a hydrogen atom or an alkyl group such that the sum of the carbon atoms in R₁₄ and R₁₅ does not exceed 10.

The polymers comprising such units can also comprise units derived from non-zwitterionic monomers such as dimethyl- or diethylaminoethyl acrylate or methacrylate or alkyl acrylates or methacrylates, acrylamides or methacrylamides or vinyl acetate.

By way of example, mention may be made of the copolymers of methyl methacrylate/methyl dimethylcarboxymethylammonioethyl methacrylate such as the product sold under the name DIAFORMER Z301 by the company Sandoz.

(5) polymers derived from chitosan comprising monomer units corresponding to the following formulae:

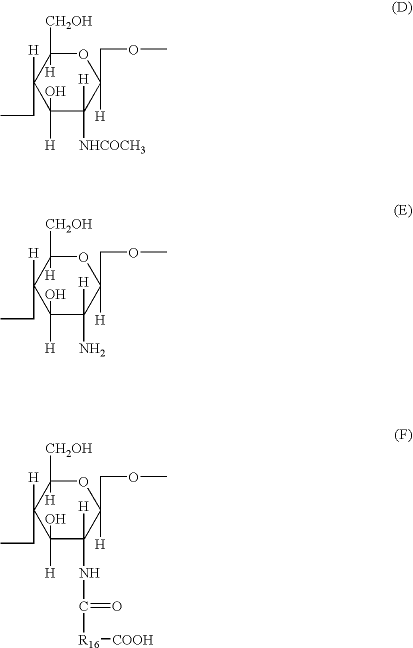

the unit (D) being present in proportions ranging from 0 to 30%, the unit (E) in proportions ranging from 5% to 50% and the unit (F) in proportions ranging from 30% to 90%, it being understood that, in this unit (F), R₁₆ represents a group of formula:

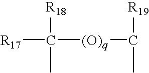

in which, if q=0, R₁₇, R₁₈ and R₁₉, which may be identical or different, each represent a hydrogen atom, a methyl, hydroxyl, acetoxy, or amino residue, a monoalkylamine residue or a dialkylamine residue that are optionally interrupted by at least one nitrogen atom and/or optionally substituted with at least one amine, hydroxyl, carboxyl, alkylthio, or sulphonic groups, an alkylthio residue in which the alkyl group bears an amino residue, at least one of the groups R₁₇, R₁₈ and R₁₉ being, in this case, a hydrogen atom; or, if q=1, R₁₇, R₁₈ and R₁₉ each represent a hydrogen atom, as well as the salts formed by these compounds with bases or acids.

(6) polymers with units corresponding to the general formula (V) are described, for example, in French patent 1 400 366:

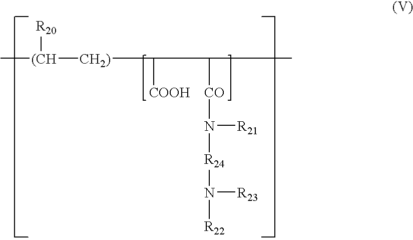

in which R₂₀ represents a hydrogen atom, a CH₃O, CH₃CH₂O or phenyl group, R₂₁ represents a hydrogen atom or a lower alkyl group such as methyl or ethyl, R₂₂ represents a hydrogen atom or a C₁₋₆ lower alkyl group such as methyl or ethyl, R₂₃ represents a C₁₋₆ lower alkyl group such as methyl or ethyl or a group corresponding to the formula: —R₂₄—N(R₂₂)₂, R₂₄ representing a —CH₂—CH₂—, —CH₂—CH₂—CH₂— or —CH₂—CH(CH₃)— group, R₂₂ having the meanings mentioned above.

(7) polymers derived from the N-carboxyalkylation of chitosan, such as N-carboxymethylchitosan or N-carboxybutylchitosan sold under the name “EVALSAN” by the company Jan Dekker.

(8) amphoteric polymers of the type -D-X-D-X chosen from:
a) polymers obtained by the action of chloroacetic acid or sodium chloroacetate on compounds comprising at least one unit of formula:

-D-X-D-X-D- (VI)

where D represents a group

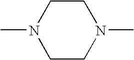

and X represents the symbol E or E′, E or E′, which may be identical or different, represent a divalent group that is an alkylene group with a straight or branched chain comprising up to 7 carbon atoms in the main chain, which is unsubstituted or substituted with hydroxyl groups and which can comprise, in addition to oxygen, nitrogen and sulphur atoms, 1 to 3 aromatic and/or heterocyclic rings; the oxygen, nitrogen and sulphur atoms may be present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups;
b) polymers of formula:

-D-X-D-X- (VI′)

where D represents a group

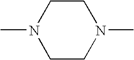

and X represents the symbol E or E′ and at least once E′; E having the meaning given above and E′ is a divalent group that is an alkylene group with a straight or branched chain having up to 7 carbon atoms in the main chain, which is unsubstituted or substituted with at least one hydroxyl group and comprising at least one nitrogen atom, the nitrogen atom being substituted with an alkyl chain that is optionally interrupted by an oxygen atom and possibly comprising at least one carboxyl function or at least one hydroxyl function and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also comprise other vinyl comonomers such as vinylcaprolactam.

Among the amphoteric fixing polymers mentioned above, the ones that may be used according to the disclosure are those of family (3), such as the copolymers whose CTFA name is octylacrylamide/acrylates/butylaminoethyl methacrylate copolymer, such as the products sold under the names AMPHOMER®, AMPHOMER® LV 71 or LOVOCRYL® 47 by the company National Starch and those of family (4) such as the copolymers of methyl methacrylate/methyl dimethylcarboxymethylammonioethyl methacrylate, sold, for example, under the name DIAFORMER Z301 by the company Sandoz.

The non-ionic fixing polymers that may be used according to the present disclosure are chosen, for example, from:

polyalkyloxazolines;

vinyl acetate homopolymers;

vinyl acetate copolymers, for instance copolymers of vinyl acetate and of acrylic ester, copolymers of vinyl acetate and of ethylene, or copolymers of vinyl acetate and of maleic ester, for example of dibutyl maleate;

homopolymers and copolymers of acrylic esters, for instance copolymers of alkyl acrylates and of alkyl methacrylates, such as the products sold by the company Rohm & Haas under the names PRIMAL® AC-261 K and EUDRAGIT® NE 30 D, by the company BASF under the name 8845, or by the company Hoechst under the name APPRETAN® N9212;

copolymers of acrylonitrile and of a non-ionic monomer chosen, for example, from butadiene and alkyl(meth)acrylates; mention may be made of the products sold under the name CJ 0601 B by the company Rohm & Haas;

styrene homopolymers;

styrene copolymers, for instance copolymers of styrene and of an alkyl (meth)acrylate, such as the products MOWILITH® LDM 6911, MOWILITH® DM 611 and MOWILITH® LDM 6070 sold by the company Hoechst, and the products RHODOPAS® SD 215 and RHODOPAS® DS 910 sold by the company Rhone-Poulenc; copolymers of styrene, of alkyl methacrylate and of alkyl acrylate; copolymers of styrene and of butadiene; or copolymers of styrene, of butadiene and of vinylpyridine;

polyamides;

vinyllactam homopolymers such as vinylpyrrolidone homopolymers and such as the polyvinylcaprolactam sold under the name LUVISKOL® Plus by the company BASF; and

vinyllactam copolymers such as a poly(vinylpyrrolidone/vinyllactam) copolymer sold under the trade name LUVITEC® VPC 55K65W by the company BASF, poly(vinylpyrrolidone/vinyl acetate) copolymers, such as those sold under the name PVPVA® S630L by the company ISP, LUVISKOL® VA 73, VA 64, VA 55, VA 37 and VA 28 by the company BASF; and poly(vinylpyrrolidone/vinyl acetate/vinyl propionate) terpolymers, for instance the product sold under the name LUVISKOL® VAP 343 by the company BASF.

The alkyl groups of the non-ionic polymers mentioned above may comprise from 1 to 6 carbon atoms.

According to the disclosure, it is also possible to use fixing polymers of grafted silicone type comprising a polysiloxane portion and a portion consisting of a non-silicone organic chain, one of the two portions constituting the main chain of the polymer, and the other being grafted onto said main chain.

These polymers are described, for example, in patent applications EP-A-0 412 704, EP-A-0 412 707, EP-A-0 640 105 and WO 95/00578, EP-A-0 582 152 and WO 93/23009 and U.S. Pat. Nos. 4,693,935, 4,728,571 and 4,972,037.

These polymers may be amphoteric, anionic or non-ionic, and are for example anionic or non-ionic.

Such polymers are, for example, copolymers that can be obtained by free radical polymerization from the monomer mixture formed from:

a) 50 to 90% by weight of tert-butyl acrylate;

b) 0 to 40% by weight of acrylic acid;

c) 5 to 40% by weight of a silicone macromer of formula:

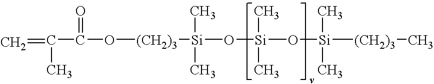

in which v is a number ranging from 5 to 700, the weight percentages being calculated relative to the total weight of the monomers.

Other examples of grafted silicone polymers may be, polydimethylsiloxanes (PDMSs) onto which are grafted, via a thiopropylene-type connecting chain, mixed polymer units of the poly(meth)acrylic acid type and of the polyalkyl(meth)acrylate type and polydimethylsiloxanes (PDMSs) onto which are grafted, via a thiopropylene-type connecting chain, polymer units of the polyisobutyl(meth)acrylate type.

Another type of silicone fixing polymer that may be mentioned is the product LUVIFLEX® Silk, sold by the company BASF.

Functionalized or non-functionalized, silicone or non-silicone, cationic, non-ionic, anionic or amphoteric polyurethanes or mixtures thereof may also be used as fixing polymers.

For example, the polyurethanes intended by the present disclosure are those disclosed in patent applications EP 0 751 162, EP 0 637 600, EP 0 648 485 and FR 2 743 297, of which the Applicant is the Proprietor, and patent applications EP 0 656 021 and WO 94/03510 from the company BASF and EP 0 619 111 from the company National Starch.

As polyurethanes that may be suitable for the present disclosure, mention may be made of the products sold under the names LUVISET PUR® and LUVISET® Si Pur by the company BASF.

The concentration of the at least one additional fixing polymer that may used in the compositions according to the present disclosure ranges from 0.1% to 20% by weight relative to the total weight of the composition, such as an amount ranging from 0.5% to 10% by weight.

A person skilled in the art should know how to add the additives without disturbing the properties of the compositions of the disclosure.

The compositions may be in the form of gels. The compositions may have a viscosity greater than 500 cps at a temperature of 25° C. and at a shear rate of 1 s−1.

When the composition according to the disclosure is packaged in an aerosol device, it comprises at least one propellant, which may be chosen from volatile hydrocarbons, such as N-butane, propane, isobutane, and pentane, halogenated hydrocarbons and mixes thereof. It is also possible to use carbon dioxide, nitrous oxide, dimethyl ether (DME), nitrogen or compressed air as the propellant. Mixtures of propellants can also be used. For example, dimethyl ether may be used.

The propellant may be present at a concentration ranging from 5 to 90% by weight relative to the total weight of the composition in the aerosol device, such as at a concentration ranging from 10 to 60% by weight.

The composition according to the disclosure may be used as a leave-in application on the hair.

Another subject of the disclosure is a method for shaping the hair, comprising the application of a cosmetic composition according to the disclosure. For example, the disclosure relates to a styling method comprising the application of a composition according to the disclosure to the hair, optional rinsing of the hair, then the shaping and drying of the hair.

The examples that follow illustrate the disclosure without limiting the scope thereof.

### EXAMPLES

### Example 1

The following compositions are produced:

The concentrations are expressed as grams of active material per 100 g of composition.

### As AM Percentage

| INCI name | A | B | C |
|---|---|---|---|
| Branched sulphonic polyester | 3.8 | 2.85 | 4.2 |
| (1) | | | |
| Polyacrylate-3 (2) | — | 0.22 | — |
| Amodimethicone (and) | 0.28 | — | 0.44 |
| Cetrimonium chloride (and) | | | |
| Trideceth-10 (3) | | | |
| Amodimethicone (and) | — | 0.24 | — |
| Trideceth-6 (and) | | | |
| Cetrimonium chloride (4) | | | |
| Propylene glycol | — | 0.38 | — |
| Glycerol | — | — | 3.3 |
| Laureth-4 (5) | 0.76 | 0.35 | — |
| Steareth-2 (6) | — | — | 0.42 |
| PEG-40 hydrogenated castor | 0.47 | 0.9 | 1.25 |
| oil (7) | | | |
| Cetrimonium chloride (8) | — | 0.05 | 0.1 |
| Cocamidopropylbetaine (and) | 0.2 | — | — |
| glyceryl laurate (9) | | | |
| Triethanolamine | — | 0.05 | — |
| Preservatives | 0.165 qs | 0.67 qs | 0.45 qs |
| Fragrance | 0.2850 | 0.38 | 0.28 |
| Denatured alcohol | — | — | 4.75 |
| Isobutane (and) Butane (and) | 5 | 5 | 5 |
| Propane (10) | | | |
| Water | qs for 100 g | qs for 100 g | qs for 100 g |

(1) EASTMAN AQ 1350
(2) VISCOPHOBE DB1000 (Dow Chemical)
(3) BELSIL ADM 6057E (Wacker)
(4) DOW CORNING 2-8299 cationic emulsion (Dow Corning)
(5) BRIJ 30 (Croda)
(6) BRIJ 72 (Croda)
(7) CREMOPHOR CO 40 (Basf)
(8) DEHYQUART A OR (Cognis)
(9) TEGOBETAINE HS (Evonik Goldschmidt)
(10) PROPEL 45 (Repsol)

The compositions are packaged in suitable aerosol containers. These compositions are easily distributed over the hair and fix said hair with good shape retention.

### Comparative Example 2

| | | E (outside the |
|---|---|---|
| | D (inventive) | disclosure) |
| Branched sulphonic | 2.85 | 2.85 |
| polyester (1) | | |
| Polyacrylate-3 (2) | 0.22 | 0.22 |
| Laureth-4 (5) | 0.76 | — |
| Decyl glucoside (11) | — | 0.76 |
| Triethanolamine | 0.047 | 0.047 |
| ISOBUTANE (and) | 5 | 5 |
| BUTANE (and) | | |
| PROPANE (10) | | |
| Water | qs for 100 | qs for 100 |

(11) Oramix CG 110 (Seppic)

The compositions are sprayed onto the hair. The mousse D expands rapidly and silently whereas the mousse E is noisy and liquid on exiting before expanding. Furthermore the mousse E soaps on locks on application, which the mousse D does not do.

What is claimed is:

1. A cosmetic composition comprising, in a cosmetically acceptable medium:
(i) at least one branched sulphonic polyester, and
(ii) at least one non-ionic oxyalkylenated or glycerolated surfactant;
wherein the oxyalkylenated or glycerolated surfactant is a compound comprising at least one hydrocarbon-based chains comprising at least 6 carbon atoms and at least one group of structure:
—CH₂—(C(H)ₜ(CH₂R₁)ₙ)_{q}—CH₂p-O—
wherein n, p, and q represent, independently of one another, 0 or 1;
t represents 1 or 2; and
R₁ represents a hydrogen atom or a hydroxy radical; and
wherein the composition comprises at least one non-ionic oxyalkylenated or glycerolated surfactant in an amount ranging from 0.1 to 3% by weight relative to the total weight of the composition.

2. A cosmetic composition according to claim 1, wherein the at least one branched sulphonic polyester is obtained by polycondensation of:
(a) at least one dicarboxylic acid that does not bear a sulphonic functional group,
(b) at least one diol or a mixture of a diol and of a diamine,
(c) at least one monomer comprising two identical or different reactive functional groups chosen from hydroxyl, amino, and carboxyl groups, and at least one sulphonic functional group; and
(d) at least one monomer comprising at least three identical or different reactive functional groups chosen from hydroxyl, amino, and carboxyl groups.

3. A cosmetic composition according to claim 2, wherein the at least one branched sulphonic polyester comprises units (e) derived from monomers comprising two different reactive functional groups chosen from hydroxy carboxylic acids and amino carboxylic acids or mixtures thereof.

4. A cosmetic composition according to claim 1, wherein the at least one branched sulphonic polyester is present in an amount ranging from 0.2 to 15% relative to the total weight of the composition.

5. A cosmetic composition according to claim 1, wherein the at least one branched sulphonic polyester is present in an amount ranging from 0.5 to 10% relative to the total weight of the composition.

6. A cosmetic composition according to claim 1, wherein the at least one non-ionic oxyalkylenated or glycerolated surfactant is chosen from:
oxyalkylenated or glycerolated fatty alcohols;
oxyalkylenated alkylphenols, the alkyl chain of which is a C₈-C₁₈ alkyl chain;
oxyalkylenated or glycerolated fatty amides;
oxyalkylenated vegetable oils;
oxyalkylenated sorbitan esters of C₆-C₃₀ acids;
oxyalkylenated sucrose esters of fatty acids;
polyethylene glycol esters of fatty acids;
copolymers of ethylene oxide and of propylene oxide; and
mixtures thereof.

7. A cosmetic composition according to claim 1, wherein the mean number of the at least one non-ionic oxyalkylene unit present in the composition ranges from 2 to 150 units.

8. A cosmetic composition according to claim 1, wherein the at least one non-ionic oxyalkylene unit is chosen from oxyethylene and oxypropylene units or mixtures thereof.

9. A cosmetic composition according to claim 1, further comprising at least one additional fixing polymer chosen from anionic, non-ionic, amphoteric, or cationic fixing polymers.

10. A cosmetic composition according to claim 1, further comprising at least one adjuvant chosen from thickening agents, penetrants, fragrances, dyes, plasticizers, buffers, ceramides, pseudoceramides, vitamins or provitamins, opacifiers, reducing agents, emulsifiers, preservatives, mineral fillers, pearlescent agents, flakes, sunscreens, proteins, moisturizers, emollients, demulcents, anti-foaming agents, antiperspirants, free-radical scavengers, bactericides, sequestrants, anti-dandruff agents, antioxidants, basifying agents, acidifying agents surfactants other than those of the disclosure; and silicones.

11. A cosmetic composition according claim 10, wherein the at least one adjuvant is panthenol.

12. A method for styling or shaping keratin materials comprising applying the cosmetic composition according to claim 1 to the keratin materials.

13. A method according to claim 12, wherein the keratin materials are chosen from human hair.

14. A method for styling or shaping hair comprising
(a) applying the composition according to claim 1 to the hair;
(b) optional rinsing of the hair, and
(c) shaping and drying of the hair.
